(19) United States
(12) Patent Application Publication
(10) Pub. No.: US 2008/0076801 A1
(43) Pub. Date: Mar. 27, 2008

(54) Indolylalkylpyridin-2-amines for the inhibition of beta-secretase

(75) Inventors: Yinfa Yan, Bedminster, NJ (US); Ping Zhou, Plainsboro, NJ (US); Yi Fan, Robbinsville, NJ (US); Albert Jean Robichaud, Ringoes, NJ (US); Michael Sotirios Malamas, Jamison, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/903,073

(22) Filed: Sep. 20, 2007

Related U.S. Application Data
(60) Provisional application No. 60/846,373, filed on Sep. 21, 2006.

(51) Int. Cl. A61K 31/4375; A61P 25/28; C07D 213/02

(52) U.S. Cl. 514/339; 546/277.4

(57) ABSTRACT

The present invention provides an indolylalkylpyridin-2-amine compound of formula I 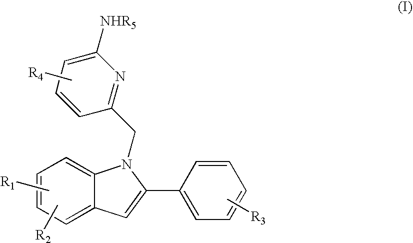 The present invention also provides methods for the use thereof to inhibit β-secretase (BACE) and treat β-amyloid deposits and neurofibrillary tangles.

This application claims the benefit under 35 U.S.C. §119(e) to co-pending U.S. provisional application Ser. No. 60/846,373, filed Sep. 21, 2006, which is hereby incorporated by reference in its entirety.

### BACKGROUND

β-Amyloid deposits and neurofibrillary tangles are two major pathologic characterizations associated with Alzheimer's disease (AD). Clinically, AD is characterized by the loss of memory, cognition, reasoning, judgment, and orientation. Also affected, as the disease progresses, are motor, sensory, and linguistic abilities until global impairment of multiple cognitive functions occurs. These cognitive losses take place gradually, but typically lead to severe impairment and eventual death in 4-12 years.

Amyloidogenic plaques and vascular amyloid angiopathy also characterize the brains of patients with Trisomy 21 (Down's Syndrome), Hereditary Cerebral Hemorrhage with Amyloidosis of the Dutch-type (HCHWA-D), and other neurodegenerative disorders. Neurofibrillary tangles also occur in other neurodegenerative disorders including dementia-inducing disorders (Varghese, J., et al, Journal of Medicinal Chemistry, 2003, 46, 4625-4630).

β-amyloid deposits are predominately an aggregate of Aβ peptide, which in turn is a product of the proteolysis of amyloid precursor protein (APP). More specifically, Aβ peptide results from the cleavage of APP at the C-terminus by one or more γ-secretases, and at the N-terminus by β-secretase enzyme (Beta-site APP Cleaving Enzyme or BACE), also known as aspartyl protease, as part of the β-amyloidogenic pathway.

BACE activity is correlated directly to the generation of Aβ peptide from APP (Sinha, et al, Nature, 1999, 402, 537-540), and studies increasingly indicate that the inhibition of BACE inhibits the production of Aβ peptide (Roberds, S. L., et al, Human Molecular Genetics, 2001, 10, 1317-1324).

Therefore, it is an object of this invention to provide compounds which are inhibitors of β-secretase and are useful as therapeutic agents in the treatment, prevention or amelioration of a disease or disorder characterized by elevated β-amyloid deposits or β-amyloid levels in a patient.

It is another object of this invention to provide therapeutic methods and pharmaceutical compositions useful for the treatment, prevention or amelioration of a disease or disorder characterized by elevated β-amyloid deposits or β-amyloid levels in a patient.

It is a feature of this invention that the compounds provided may also be useful to further study and elucidate the β-secretase enzyme.

These and other objects and features of the invention will become more apparent by the detailed description set forth hereinbelow.

### SUMMARY OF THE INVENTION

The present invention provides a compound of formula I 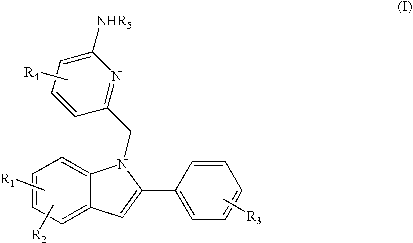
wherein
R₁ and R₂ are each independently H, halogen, CN, OR₆, CO₂R₇, COR₈, NR₁₁R₁₂ or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₃ is H, halogen or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₄ is H, halogen, NR₁₃R₁₄, OR₁₅ or a C₁-C₆alkyl or aryl group each group optionally substituted;
R₅ is H or an optionally substituted C₁-C₆alkyl group;
R₆ is H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₇, R₈ and R₁₅ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted; and
R₁₁, R₁₂, R₁₃ and R₁₄ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted or R₁₁R₁₂ or R₁₃R₁₄ may be taken together with the atom to which they are attached to form an optionally substituted 5- to 7-membered ring optionally containing an additional heteroatom selected from O, N or S; or
   a stereoisomer thereof or a pharmaceutically acceptable salt thereof.

The present invention also relates to the use of such compounds, where R₆ is other than H, for the treatment of β-amyloid deposits and neurofibrillary tangles. These formula I compounds are particularly useful in treating Alzheimer's disease, cognitive impairment, Down's Syndrome, HCHWA-D, cognitive decline, senile dementia, cerebral amyloid angiopathy, degenerative dementia, or other neurodegenerative disorders.

### DETAILED DESCRIPTION OF THE INVENTION

Alzheimer's disease (AD) is a major degenerative disease of the brain which presents clinically by progressive loss of memory, cognition, reasoning, judgement and emotional stability and gradually leads to profound mental deterioration and death. The exact cause of AD is unknown, but increasing evidence indicates that amyloid beta peptide (A-beta) plays a central role in the pathogenesis of the disease. (D. B. Schenk; R. E. Rydel et al, Journal of Medicinal Chemistry, 1995, 21,4141 and D. J. Selkoe, Physiology Review, 2001, 81, 741). Patients with AD exhibit characteristic neuropathological markers such as neuritic plaques (and in β-amyloid angiopathy, deposits in cerebral blood vessels) as well as neurofibrillary tangles detected in the brain at autopsy. A-beta is a major component of neuritic plaques in AD brains. In addition, β-amyloid deposits and vascular β-amyloid angiopathy also characterize individuals with Downs Syndrome, Hereditary Cerebral Hemorrhage with Amyloidosis of the Dutch type and other neurodegenerative and dementia-inducing disorders. Overexpression of the amyloid precursor protein (APP), altered cleavage of APP to A-beta or a decrease in the clearance of A-beta from a patient's brain may increase the levels of insoluble or fibrillar forms of A-beta in the brain. The β-site APP cleaving enzyme, BACE1, also called memapsin-2 or Asp-2, was identified in 1999 (R. Vassar, B. D. Bennett, et al, Nature, 1999, 402, 537). BACE1 is a membrane-bound aspartic protease with all the known functional properties and characteristics of β-secretase. Low molecular weight, non-peptide, non-substrate-related inhibitors of BACE1 or β-secretase are earnestly sought both as an aid in the study of the β-secretase enzyme and as potential therapeutic agents.

Surprisingly, it has now been found that indolylalkylpyridin-2-amine compounds of formula I wherein R₆ is other than H demonstrate inhibition of β-secretase. Advantageously, said pyridin-2-amine compounds may be used as effective therapeutic agents for the treatment, prevention or amelioration of a disease or disorder characterized by elevated β-amyloid deposits or β-amyloid levels in a patient. Compounds of formula I wherein R₆ is H are useful as intermediates in the manufacture of said therapeutic agents. Accordingly, the present invention provides an indolylalkylpyridin-2-amine compound of formula I 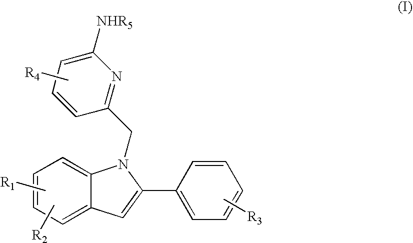
wherein
R₁ and R₂ are each independently H, halogen, CN, OR₆, CO₂R₇, COR₈, NR₁₁R₁₂ or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₃ is H, halogen or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₄ is H, halogen, NR₁₃R₁₄, OR₁₅ or a C₁-C₆alkyl or aryl group each group optionally substituted;
R₅ is H or an optionally substituted C₁-C₆alkyl group;
R₆ is H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₇, R₈ and R₁₅ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted; and
R₁₁, R₁₂, R₁₃ and R₁₄ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted or R₁₁R₁₂ or R₁₃R₁₄ may be taken together with the atom to which they are attached to form an optionally substituted 5- to 7-membered ring optionally containing an additional heteroatom selected from O, N or S; or
   a stereoisomer thereof or a pharmaceutically acceptable salt thereof.

It is understood that the claims encompass all possible stereoisomers and prodrugs.

An optionally substituted moiety may be substituted with one or more substituents. The substituent groups which are optionally present may be one or more of those customarily employed in the development of pharmaceutical compounds or the modification of such compounds to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylamido, aryl (e.g. phenyl), aryloxy (e.g. phenoxy), arylalkyl (e.g. benzyl), arylalkyloxy (e.g. benzyloxy), heteroaryl, heterocycloalkyl or cycloalkyl groups, wherein the aryl (e.g. phenyl) in any of the above is further optionally substituted with one or more halo, cyano, alkyl, haloalkyl or alkoxy groups. Unless otherwise specified, typically, 0-4 substituents may be present. When any of the foregoing substituents represents or contains an alkyl substituent group, this may be linear or branched and may contain up to 12 carbon atoms, preferably up to 6 carbon atoms, more preferably up to 4 carbon atoms. Preferably an optionally substituted moiety may be substituted with one or more, e.g. 1-3, substituents selected from the group consisting of halo, cyano, hydroxyl, C₁-C₆alkyl, C₁-C₆haloalkyl, C₁-C₆alkoxy, or C₆-C₁₄aryl, wherein the aryl is further optionally substituted with one or more halo, cyano, C₁-C₄alkyl, C₁-C₄haloalkyl or C₁-C₄alkoxy groups.

As used herein, the term “alkyl” includes both a straight chain and a branched-chain monovalent saturated hydrocarbon moiety, e.g. of one to twelve carbon atoms, preferably one to six carbon atoms, more preferably one to four carbon atoms. Examples of saturated hydrocarbon alkyl moieties include, but are not limited to, chemical groups such as methyl, ethyl, n-propyl, isopropyl, n-butyl, tert-butyl, isobutyl, sec-butyl; higher homologs such as n-pentyl, n-hexyl, and the like.

The term “alkenyl”, as used herein, refers to an alkyl group as defined above containing one or more carbon-carbon double bonds. Alkenyl groups preferably contain two to six carbon atoms. Such alkenyl moieties may be mono or polyunsaturated, and may exist in the E or Z configurations. The compounds of this invention are meant to include all possible E and Z configurations. Examples of mono or polyunsaturated alkenyl moieties include, but are not limited to, vinyl, 2-propenyl, isopropenyl, crotyl, 2-isopentenyl, butadienyl, 2-(butadienyl), 2,4-pentadienyl, 3-(1,4-pentadienyl), and higher homologs, isomers, or the like. In some embodiments, alkenyl groups can be substituted with one to three substituent groups, as described hereinabove.

The term “alkynyl”, as used herein, refers to an alkyl group as defined above having one or more triple carbon-carbon bonds. Alkynyl groups preferably contain 2 to 6 carbon atoms. Examples of alkynyl groups include, but are not limited to, ethynyl, propynyl, butynyl, pentynyl, and the like. In some embodiments, alkynyl groups can be substituted with up to three substituent groups, as described hereinabove.

The term “cycloalkyl”, as used herein, refers to a monocyclic, bicyclic, tricyclic, fused, bridged, or spiro saturated hydrocarbon moiety of 3-10 carbon atoms, preferably 3-8 carbon atoms. Any suitable ring position of the cycloalkyl moiety may be covalently linked to the defined chemical structure. Examples of cycloalkyl moieties include, but are not limited to, chemical groups such as cyclopropyl, cyclobutyl, cyclopentyl, cyclohexyl, cycloheptyl, norbornyl, adamantyl, spiro[4.5]decanyl, and homologs, isomers, or the like. In some embodiments, cycloalkyl groups can be substituted with up to three substituent groups, as described hereinabove.

The term “cycloheteroalkyl” as used herein designates a 3- to 8-membered cycloalkyl ring system, e.g. a 5- to 7-membered ring system, wherein 1, 2 or 3 of the carbon atoms are replaced by heteroatoms, which may be the same or different, selected from N, O or S, and optionally containing one double bond. Exemplary of the cycloheteroalkyl ring systems included in the term as designated herein are the following rings wherein X is NR, O or S and R is H or an optional substituent as defined hereinbelow.

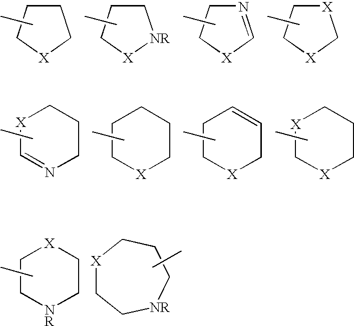

The term “aryl”, as used herein, designates an aromatic carbocyclic moiety of up to 20 carbon atoms, e.g. 6-20 carbon atoms, preferably 6-14 carbon atoms, which may be a single ring (monocyclic) or multiple rings (e.g. bicyclic or tricyclic) fused together or linked covalently, wherein at least one of the multiple rings is aromatic. Any suitable ring position of the aryl moiety may be covalently linked to the defined chemical structure. Examples of aryl moieties include, but are not limited to, chemical groups such as phenyl, 1-naphthyl, 2-naphthyl, biphenyl, anthryl, phenanthryl, fluorenyl, indanyl, and the like. In some embodiments “aryl” groups can be substituted with up to three substituent groups, as defined hereinabove.

The term “heteroaryl” as used herein designates an aromatic heterocyclic ring system e.g. having from 5-20 ring members, which may be a single ring (monocyclic) or multiple rings (e.g. bicyclic or tricyclic) fused together or linked covalently, wherein at least one of the multiple rings is aromatic. Preferably, heteroaryl is a 5- or 6-membered ring. The rings may contain from one to four hetero atoms selected from nitrogen, oxygen, or sulfur, wherein the nitrogen or sulfur atom(s) are optionally oxidized, or the nitrogen atom(s) are optionally quarternized. Any suitable ring position of the heteroaryl moiety may be covalently linked to the defined chemical structure. Examples of heteroaryl moieties include, but are not limited to chemical groups such as furan, thiophene, pyrrole, N-methylpyrrole, pyrazole, N-methylpyrazole, imidazole, N-methylimidazole, oxazole, isoxazole, thiazole, isothiazole, 1H-tetrazole, 1-methyltetrazole, 1,3,4-oxadiazole, 1H-1,2,4-triazole, 1-methyl-1,2,4-triazole 1,3,4-triazole, 1-methyl-1,3,4-triazole, pyridine, pyrimidine, pyrazine, pyridazine, benzoxazole, benzisoxazole, benzothiazole, benzofuran, benzothiophene, thianthrene, dibenzo[b,d]furan, dibenzo[b,d]thiophene, benzimidazole, N-methylbenzimidazole, indole, indazole, quinoline, isoquinoline, quinazoline, quinoxaline, purine, pteridine, 9H-carbazole, α-carboline, or the like.

The term “halogen”, as used herein, designates fluorine, chlorine, bromine, or iodine.

The compounds of the present invention may be converted to salts, in particular pharmaceutically acceptable salts using art recognized procedures. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Salts which are unsuitable for pharmaceutical uses but which can be employed, for example, for the isolation or purification of free compounds or their pharmaceutically acceptable salts, are also included. The term “pharmaceutically acceptable salt”, as used herein, refers to salts derived form organic and inorganic acids such as, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety. Salts may also be formed from organic and inorganic bases, preferably alkali metal salts, for example, sodium, lithium, or potassium, when a compound of this invention contains a carboxylate or phenolic moiety, or similar moiety capable of forming base addition salts.

The compounds of this invention may contain an asymmetric carbon atom and some of the compounds of this invention may contain one or more asymmetric centers and may thus give rise to optical isomers and diastereomers. While shown without respect to stereochemistry in formula I, the present invention includes such optical isomers and diastereomers; as well as the racemic and resolved, enantiomerically pure R and S stereoisomers; as well as other mixtures of the R and S stereoisomers and pharmaceutically acceptable salts thereof. Where a stereoisomer is preferred, it may in some embodiments be provided substantially free of the corresponding enantiomer. Thus, an enantiomer substantially free of the corresponding enantiomer refers to a compound that is isolated or separated via separation techniques or prepared free of the corresponding enantiomer. “Substantially free”, as used herein, means that the compound is made up of a significantly greater proportion of one stereoisomer, preferably not less than about 50%, more preferably not less than about 75%, and even more preferably not less than about 90%.

Preferred compounds of formula I are those compounds wherein R₄ and R₅ are H. Another group of preferred compounds are those compounds of formula I wherein R₃ is H or halogen. Also preferred are those compounds of formula I wherein R₁ is OR₆ and R₂ is H.

More preferred compounds of the invention are those compounds of formula I wherein R₄ and R₅ are H and R₃ is halogen. Another group of more preferred compounds of the invention are those compounds of formula I wherein R₁ is OR₆; R₂ is H; and R₆ is an optionally substituted C₁-C₆alkyl group or heteroaryl. A further group of more preferred compounds of the invention are those compounds of formula I wherein R₁ is OR₆; R₂ is H; R₃ is halogen; and R₃ is in the 2-position.

Preferred compounds of the invention include:
4-{[1-[(6-aminopyridin-2-yl)methyl]-2-(2-chlorophenyl)-1H-indol-6-yl]oxy}butanenitrile;
6-{[6-bromo-2-(2-chlorophenyl)-1H-indol-1-yl]methyl}pyridin-2-amine;
6-{[6-(benzyloxy)-2-(2-chlorophenyl)-1H-indol-1-yl]methyl}pyridin-2-amine;
6-({2-(2-chlorophenyl)-6-[(3-fluorobenzyl)oxy]-1H-indol-1-yl}methyl)pyridin-2-amine;
3-({[1-[(6-aminopyridin-2-yl)methyl]-2-(2-chlorophenyl)-1H-indol-6-yl]oxy}methyl)benzonitrile;
6-({2-(2-chlorophenyl)-6-[(2,5-difluorobenzyl)oxy]-1H-indol-1-yl}methyl)pyridin-2-amine;
6-[(2-(2-chlorophenyl)-6-{[3-(trifluoromethyl)benzyl]oxy}-1H-indol-1-yl)methyl]pyridin-2-amine;
6-({2-(2-chlorophenyl)-6-[(3-methoxybenzyl)oxy]-1H-indol-1-yl}methyl)pyridin-2-amine;
6-{[2-(2-chlorophenyl)-6-(pyrimidin-5-yloxy)-1H-indol-1-yl]methyl}pyridin-2-amine;
6-{[2-(2-chlorophenyl)-6-propoxy-1H-indol-1-yl]methyl}pyridin-2-amine;
6-{[2-(2-chlorophenyl)-6-methoxy-1H-indol-1-yl]methyl}pyridin-2-amine;
6-[(2-phenyl-1H-indol-1-yl)methyl]pyridin-2-amine;
4-[1-[(6-aminopyridin-2-yl)methyl]-2-(2-chlorophenyl)-1H-indol-6-yl]but-3-yn-1-ol;
5-[1-[(6-aminopyridin-2-yl)methyl]-2-(2-chlorophenyl)-1H-indol-6-yl]pent-4-yn-1-ol;
6-[1-[(6-aminopyridin-2-yl)methyl]-2-(2-chlorophenyl)-1H-indol-6-yl]hex-5-yn-1-ol;
4-[1-[(6-aminopyridin-2-yl)methyl]-2-(2-chlorophenyl)-1H-indol-6-yl]butan-1-ol;
5-[1-[(6-aminopyridin-2-yl)methyl]-2-(2-chlorophenyl)-1H-indol-6-yl]pentan-1-ol;
6-[1-[(6-aminopyridin-2-yl)methyl]-2-(2-chlorophenyl)-1H-indol-6-yl]hexan-1-ol;
6-{[2-(2-chlorophenyl)-6-(4-methoxybut-1-yn-1-yl)-1H-indol-1-yl]methyl}pyridine-2-amine;
1-[(6-aminopyridin-2-yl)methyl]-2-(2-chlorophenyl)-1H-indol-6-ol;
6-{[2-(2-chlorophenyl)-6-(6-methoxyhex-1-yn-1-yl)-1H-indol-1-yl]methyl}pyridin-2-amine;
6-{[2-(2-chlorophenyl)-6-(5-fluoropent-1-yn-1-yl)-1H-indol-1-yl]methyl}pyridin-2-amine;
6-{[2-(2-chlorophenyl)-6-(6-fluorohex-1-yn-1-yl)-1H-indol-1-yl]methyl}pyridin-2-amine;
6-{[2-(2-chlorophenyl)-6-(5-methoxypent-1-yn-1-yl)-1H-indol-1-yl]methyl}pyridin-2-amine;
6-{[2-(2-chlorophenyl)-6-(4-fluorobut-1-yn-1-yl)-1H-indol-1-yl]methyl}pyridin-2-amine;
   or a stereoisomer thereof or a pharmaceutically acceptable salt thereof.

Advantageously, the present invention provides a method for the preparation of a compound of formula I wherein R₅ is H (Ia) which comprises reacting a compound of formula II with a protected 2-aminopyridine of formula III in the presence of a base optionally in the presence of a solvent to give the protected indolylalkylpyridin-2-amine intermediate and reacting said intermediate with an acid to give the desired compound of formula Ia. The reaction is shown in flow diagram I wherein Hal represents Cl, Br or I and P represents a protecting group.

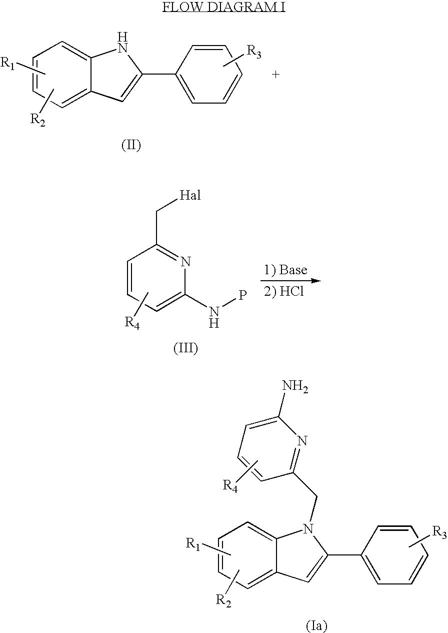

Bases suitable for use in the method of invention include Cs₂CO₃, K₂CO₃, Na₂CO₃, NaH, or any conventional base capable of removing a proton from a basic cyclic amine nitrogen atom.

Solvents suitable for use in the method of the invention include tetrahydrofuran, dimethyl formamide, dimethylsulfoxide, acetonitrile, or the like.

Protecting groups useful in the method of the invention include t-butylcarb-oxylate, benzyl, acetyl, benzyloxycarbonyl, or any conventional group known to protect a basic nitrogen in standard synthetic procedures.

Acids suitable for use in the method of invention include HCl, H₂SO4, trifluoroacetic acid, or any acid known to be useful for deprotecting a protected amine in routine synthetic procedures.

Compounds of formula II may be prepared using conventional synthetic methods and, if required, standard separation or isolation techniques. For example, compounds of formula II may be prepared by reacting a phenylhydrazine of formula IV with an acetophenone of formula V to give the phenylhydrazone of formula VI and reacting said formula VI hydrazone with poylphosphoric acid (PPA) under Fisher conditions to give the desired compound of formula II. The reaction is shown in flow diagram II.

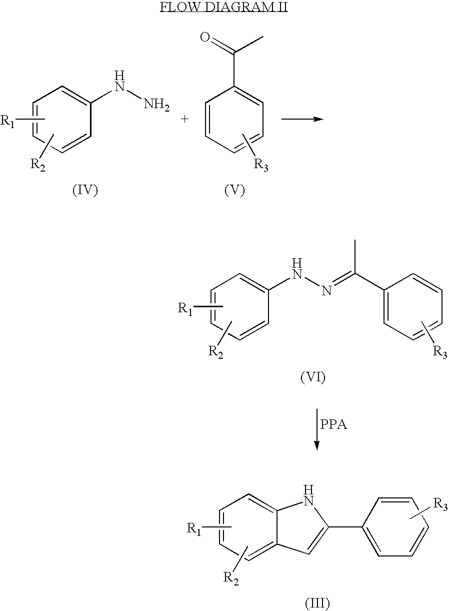

Advantageously, the compounds of formula I wherein R₆ is other than H (Ia) act as BACE inhibitors for the treatment of β-amyloid deposits and neurofibrillary tangles associated with such diseases as Alzheimer's disease, Trisomy 21 (Down's Syndrome), Hereditary Cerebral Hemorrhage with Amyloidosis of the Dutch-type (HCHWA-D), and other neurodegenerative disorders. Accordingly, the present invention provides methods for modulating BACE and treating, preventing, or ameliorating β-amyloid deposits and neurofibrillary tangles associated with diseases and disorders such as Alzheimer's disease, Trisomy 21 (Down's Syndrome), Hereditary Cerebral Hemorrhage with Amyloidosis of the Dutch-type (HCHWA-D), or other neurodegenerative disorders. Such methods include providing a patient suffering from or being susceptible to a disease or injury associated with excessive BACE activity an effective amount of a compound of formula Ia. Also according to the present invention there is provided a method of treating Alzheimer's disease and related senile dementia in humans or other mammals which comprises administering to a human or other mammal an effective amount of a compound of formula Ia.

The present invention also provides a method for the treatment of a disorder related to or associated with excessive BACE activity in a patient in need thereof which comprises providing said patient a therapeutically effective amount of at least one compound of formula Ia. Representative disorders include Alzheimer's disease, cognitive impairment, Down's Syndrome, HCHWA-D, cognitive decline, senile dementia, cerebral amyloid angiopathy, degenerative dementia, or other neurodegenerative disorders. Certain of these diseases are characterized by production of β-amyloid deposits or neurofibrillary tangles.

The present invention also provides a method for inhibiting the activity of BACE, comprising administering to a patient or contacting a receptor thereof with an effective amount of at least one compound of formula Ia. Certain methods further comprise determining BACE activity, either before or after said contacting step.

The present invention also provides a method of ameliorating β-amyloid deposits or neurofibrillary tangles in a mammal which comprises providing said mammal an effective amount of at least one compound of formula Ia.

Also provided are methods of ameliorating symptoms of Alzheimer's disease, cognitive impairment, Down's Syndrome, HCHWA-D, cognitive decline, senile dementia, cerebral amyloid angiopathy, degenerative dementia, or other neurodegenerative disorders in a mammal which comprises providing said mammal an effective amount of at least one compound of formula Ia.

Further methods prevent Alzheimer's disease, cognitive impairment, Down's Syndrome, HCHWA-D, cognitive decline, senile dementia, cerebral amyloid angiopathy, degenerative dementia, or other neurodegenerative disorders in a mammal that is known to suffer from or suspected to be at risk of suffering from such diseases. These methods comprise providing said mammal an effective amount of at least one compound of formula Ia.

As used in accordance with this invention, the term “providing,” with respect to providing a compound or substance covered by this invention, means either directly administering such a compound or substance, or administering a prodrug, derivative, or analog which will form the effective amount of the compound or substance within the body. This invention also covers providing the compounds of this invention to treat the disease states disclosed herein that the compounds are useful for treating.

The term “patient”, as used herein, refers to a mammal, preferably a human.

The terms “administer”, “administering”, or “administration”, as used herein, refer to either directly administering a compound or composition to a patient, or administering a prodrug derivative or analog of the compound to the patient, which will form an equivalent amount of the active compound or substance within the patient's body.

The terms “effective amount”, “therapeutically effective amount” and “effective dosage” as used herein, refer to the amount of a compound that, when administered to a patient, is effective to at least partially ameliorate (and, in preferred embodiments, cure) a condition from which the patient is suspected to suffer.

It is understood that the effective dosage of the active compounds of this invention may vary depending upon the particular compound utilized, the mode of administration, the condition, and severity thereof, of the condition being treated, as well as the various physical factors related to the individual being treated. For treating Alzheimer's disease and other related senile dementia's, generally, satisfactory results may be obtained when the compounds of this invention are administered to the individual in need at a daily dosage of from about 0.1 mg to about 1 mg per kilogram of body weight, preferably administered in divided doses two to six times per day, or in a sustained release form. For most large mammals, the total daily dosage is from about 3.5 mg to about 140 mg preferably from about 3.5 to about 5 mg. In the case of a 70 kg human adult, the total daily dose will generally be from about 7 mg to about 70 mg and may be adjusted to provide the optimal therapeutic result. This regimen may be adjusted to provide the optimal therapeutic response.

In one aspect, the present invention is directed to compositions comprising one or more compounds of formula Ia and one or more pharmaceutically acceptable carriers. Accordingly, the present invention provides a pharmaceutical composition which comprises a pharmaceutically acceptable carrier and a compound of formula Ia.

The term “carrier”, as used herein, shall encompass carriers, excipients, and diluents. Examples of carriers are well known to those skilled in the art and are prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and are biologically acceptable.

The formula Ia compound of the invention may be administered orally or parenterally, neat or in combination with conventional pharmaceutical carriers. Applicable solid carriers can include one or more substances which may also act as flavoring agents, lubricants, solubilizers, suspending agents, fillers, glidants, compression aids, binders or tablet-disintegrating agents or encapsulating materials. They are formulated in conventional manner, for example, in a manner similar to that used for known antihypertensive agents, diuretics and β-blocking agents. Oral formulations containing the active compounds of this invention may comprise any conventionally used oral forms, including tablets, capsules, buccal forms, troches, lozenges and oral liquids, suspensions or solutions. In powders, the carrier is a finely divided solid, which is an admixture with the finely divided active ingredient. In tablets, the active ingredient is mixed with a carrier having the necessary compression properties in suitable proportions and compacted in the shape and size desired. The powders and tablets preferably contain up to 99% of the active ingredient.

Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colliodol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or fruit juice, containing appropriate solubilizers or emulsifiers as needed.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. Such administrations may be carried out using the present compounds, or pharmaceutically acceptable salts thereof, in lotions, creams, foams, patches, suspensions, solutions, and suppositories (rectal and vaginal).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. In therapeutic application, compounds of the present invention are provided to a patient already suffering from a disease in an amount sufficient to cure or at least partially ameliorate the symptoms of the disease and its complications. An amount adequate to accomplish this is defined as a “therapeutically effective amount”. The dosage to be used in the treatment of a specific case must be subjectively determined by the attending physician. The variables involved include the specific condition and the size, age and response pattern of the patient.

In some cases it may be desirable to administer the compounds directly to the airways in the form of an aerosol. For administration by intranasal or intrabronchial inhalation, the compounds of this invention may be formulated into an aqueous or partially aqueous solution.

The active compounds of this invention may be administered parenterally or intraperitoneally. Solutions or suspensions of these active compounds as a free base or pharmaceutically acceptable salt may be prepared in water suitably mixed with a surfactant such as hydroxyl-propylcellulose. Dispersions may also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The compounds of formula Ia can be administered transdermally through the use of a transdermal patch. For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream, such as a semi-permeable membrane covering a reservoir containing the active ingredient with or without a carrier, or a matrix containing the active ingredient. Other occlusive devices are known in the literature.

The compounds of this invention of formula Ia may be administered rectally or vaginally in the form of a conventional suppository. Suppository formulations may be made from traditional materials, including cocoa butter, with or without the addition of waxes to alter the suppository's melting point, and glycerin. Water soluble suppository bases, such as polyethylene glycols of various molecular weights, may also be used.

In certain embodiments, the present invention is directed to prodrugs. Various forms of prodrugs are known in the art, for example, as discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al. (ed.), “Design and Application of Prodrugs”, Textbook of Drug Design and Development, Chapter 5, 113-191 (1991), Bundgaard, et al., Journal of Drug Deliver reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975).

It is understood that the dosage, regimen and mode of administration of these compounds will vary according to the malady and the individual being treated and will be subject to the judgment of the medical practitioner involved. It is preferred that the administration of one or more of the compounds herein begin at a low dose and be increased until the desired effects are achieved.

For a more clear understanding, and in order to illustrate the invention more clearly, specific examples thereof are set forth hereinbelow. The following examples are merely illustrative and are not to be understood as limiting the scope and underlying principles of the invention in any way.

Unless otherwise stated, all parts are parts by weight. The terms DMSO and DMF designate dimethyl sulfoxide and dimethyl formamide, respectively. The term EtOAc designates ethyl acetate. The term NMR designates proton nuclear magnetic resonance and the term MS designates mass spectroscopy with (+) referring to the positive mode which generally gives a M+1 (or M+H) absorption where M=the molecular mass. All compounds are analyzed at least by MS and NMR.

### EXAMPLE 1

### Preparation of (1E)-1-(2-Chlorophenyl)ethanone (3-methoxyphenyl)hvdrazone

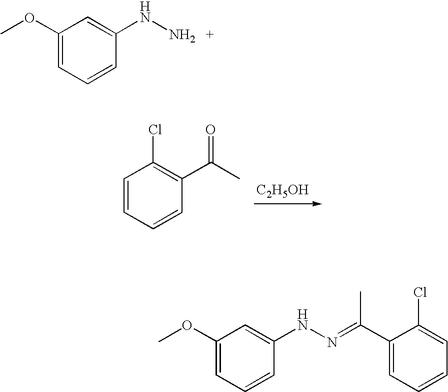

A solution of 3-methoxyphenylhydrazine (2.4 g, 17.4 mmol) and 2-choloroacetophone (2.68 g, 17.4 mmol) in ethanol is heated at reflux temperature for 24 hrs, cooled to room temperature and evaporated under reduced pressure. The resultant residue is flash chromatographed on silica gel with ethyl acetate to afford the title product as an oil, 3.2 g (67% yield); NMR (400 MHz, DMSO-d₆): δ 3.66 (s, 3H), 6.30 (d, 1H), 6.71 (m, 2H), 7.04 (t, 1H), 7.30,-7.43 (m, 4H), 9.21 (s, 1H).

### EXAMPLE 2

### Preparation of 2-(2-Chlorophenyl)-6-methoxy-1H-indole

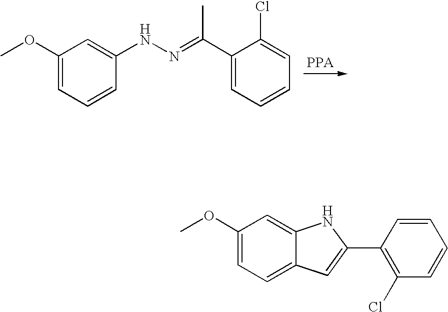

A solution of (1E)-1-(2-chlorophenyl)ethanone(3-methoxyphenyl)hydrazone of (2.8 g, 10 mmol) in xylene is added to a stirred mixture of polyphosphoric acid (PPA) in xylene at 80° C. The reaction mixture is heated to 110° C., with stirring, for 2 hrs and cooled to room temperature. The phases are separated. The organic phase is washed with water, dried over MgSO₄ and concentrated in vacuo. The resultant residue is purified by flash chromatography with ethyl acetate/hexane to afford the title product as a white solid, 1.3 g (50% yield); NMR (400 MHz, DMSO-d₆): δ 3.74 (s, 3H), 6.66 (d, 1H), 6.78 (s, 1H), 6.86 (s, 1H), 7.30 (t, 1H), 7.40 (t, 1H), 7.51 (d, 1H), 7.66 (d, 1H), 11.20 (s, 1H).

### EXAMPLE 3

### Preparation of 2,2-Dimethyl-N-(6-methylpyridin-2-yl)propanamide

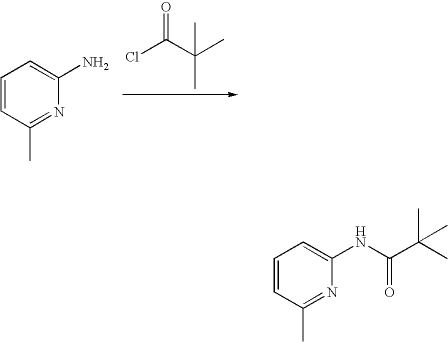

A solution of 6-methylpyridin-2-amine (5.4 g, 50 mmol) and triethylamine (5.05 g, 50 mmol) in methelene chloride at 0°-5° C. is treated with 2,2-dimethyl-propionyl chloride (6.0 g, 50 mmol) over a 30 min. period, allowed to warm to room temperature, stirred for 16 hrs and diluted with water. The phases are separated. The organic phase is dried over MgSO₄ and concentrated in vacuo. The resultant residue is purified by flash chromatography with ethyl acetate/hexane (10:90) to afford the title product 7.6 g (80%).

### EXAMPLE 4

### Preparation of N-[6-(Bromomethyl)pyridin-2-yl]-2,2-dimethylpropanamide

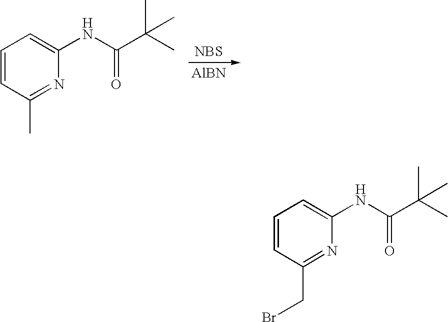

A solution of 2,2-dimethyl-N-(6-methyl-pyridin-2-yl)-propionamide (3.84 g, 20 mmol) and N-bromosuccinamide (NBS) (3.56 g, 20 mmol) in CCl₄ is treated with 2,2′-azobisisobutyronitrile (AIBN) (192 mg), heated to reflux temperature for 6 hrs and evaporated under reduced pressure. The resultant residue is flash chromatographed on silica gel with ethyl acetate/hexane (10:90) to afford the title product as an oil which solidified upon standing, 2.8 g (52% yield).

### EXAMPLE 5

### Preparation of N-(6-{[2-(2-Chlorophenyl)-6-methoxy-1H-indol-1-yl]methyl}-pyridin-2-yl)-2,2-dimethylpropanamide

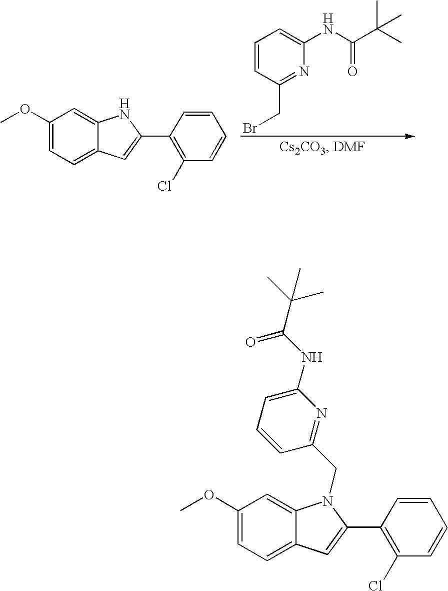

A mixture of 2-(2-chlorophenyl)-6-methoxy-1H-indole (514 mg, 2 mmol), N-(6-bromomethylpyridin-2-yl)-2,2-dimethyl-propionamide (688 mg, 2.4 mmol) and Cs₂CO₃ (780 mg, 2.4 mmol) in DMF is heated at 65° C., with stirring, for 6 hrs, cooled to room temperature and partitioned between water and ethyl acetate. The phases are separated. The organic phase dried over MgSO₄ and concentrated in vacuo. The resultant residue is purified by flash chromatography to afford the title product as a white solid, 600 mg (68% yield); NMR (400 MHz, DMSO-d₆): δ 1.18 (s, 9H), 3.71 (s, 3H), 5.23 (s, 2H), 6.00 (d, 1H), 6.55 (s, 1H), 6.78 (d, 1H), 7.00 (s, 1H), 7.36 (t, 1H), 7.40 (t, 1H), 7.51 (m, 2H), 7.80 (d, 1H), 9.60 (s, 1H).

### EXAMPLE 6

### Preparation of N-(6-{[2-(2-Chlorophenyl)-6-hydroxy-1H-indol-1-yl]methyl}pyridin-2-yl)-2,2-dimethylpropanamide

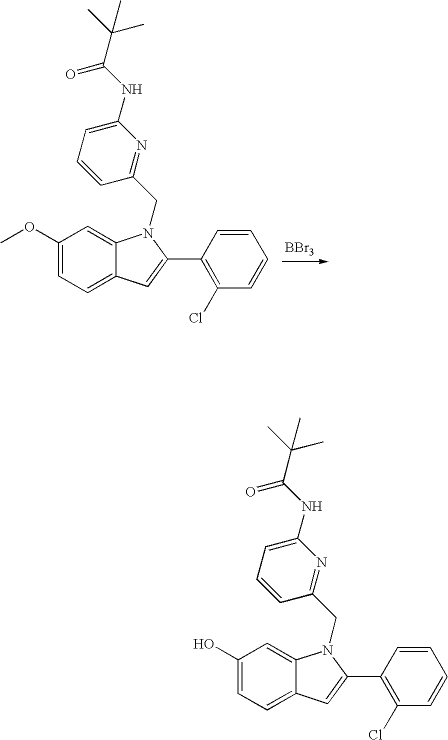

A solution of N-{6-[2-(2-chloro-phenyl)-6-methoxyindole-1-ylmethyl]pyridin-2yl}-2,2-dimethyl-propionamide (940 mg, 2 mmol) in CH₂Cl₂ at −78° C. is treated dropwise with BBr₃ (1M solution in CH₂Cl₂, 12 mL 12 mmol) over a 30 min period, allowed to warm to room temperature, stirred for 1 h at room temperature and poured into ice water. The phases are separated. The aqueous phase is extracted with CH₂Cl₂. The organic phase and the extracts are combined, dried over MgSO₄ and concentrated in vacuo to afford the title product as a white solid, 500 mg (53% yield); NMR (400 MHz, DMSO-d₆): δ 1.19 (s, 9H), 5.23 (s, 2H), 6.00 (d, 1H), 6.50 (s, 1H), 6.60 (m, 2H), 7.36 (t, 1H), 7.40 (t, 2H), 7.51 (m, 2H), 7.80 (d, 1H), 9.21 (s, 1H), 9.62 (s, 1H).

### EXAMPLE 7

### Preparation of 1-[(6-Aminopyridin-2-yl)methyl]-2-(2-chlorophenyl)-1H-indol-6-ol Hydrochloride

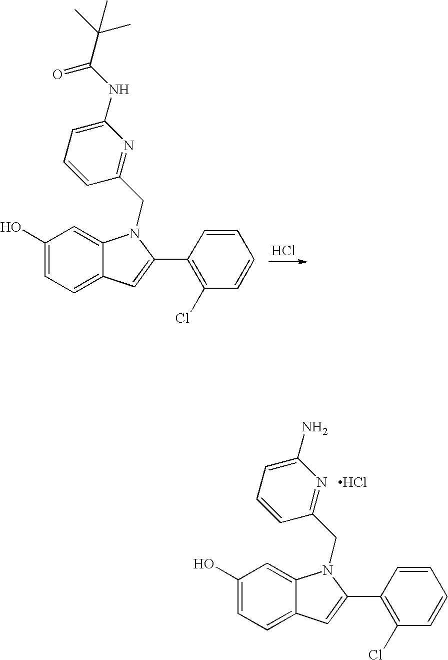

A mixture of N-{6-[2-(2-chlorophenyl)-6-hydroxyindole-1-ylmethyl]pyridin-2yl}-2,2-dimethylpropionamide (433 mg, 1 mmol) and concentrated HCl in ethanol is heated at reflux temperature for 16 h, cooled to room temperature and concentrated to dryness under reduced pressure to afford the title compound as a tan solid, 386 mg (quantitative yield); NMR (400 MHz, DMSO-d₆): δ 5.18 (s, 2H), 5.66 (d, 1H), 6.48 (s, 1H), 6.65 (d, 1H), 6.70 (s, 1H), 7.00 (s, 1H), 6.75 (t, 1H), 7.30-7.50 (m, 4H), 7.50-7.65 (m, 2H), 7.80 (b, 2H), 9.15 (b, 1H).

A solution of the tan solid in CH₂Cl₂ is washed with saturated NaHCO₃, dried over MgSO₄ and concentrated in vacuo to give the free base of the title product; MS (+) ES: 412 (M+H)⁺.

### EXAMPLE 8

### Preparation of 4-{[1-[(6-aminopyridin-2-yl)methyl]-2-(2-chlorophenyl)-1H-indol-6-yl]oxy}butanenitrile

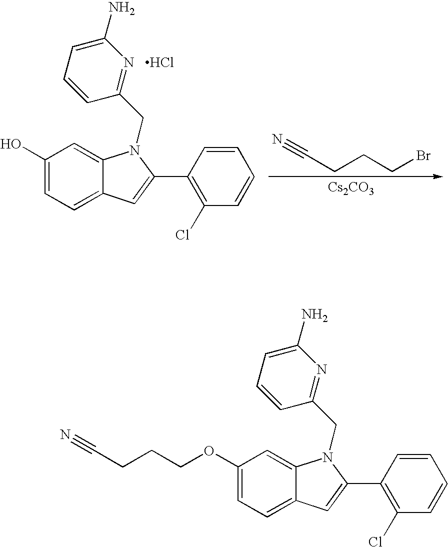

A mixture of 1-(6-aminopyridin-2ylmethyl)-2-(2-chlorophenyl)-1H-indol-6-ol hydrochloride (100 mg, 0.26 mmol), 4-bromobutyronitrile (38 mg, 0.26 Mmol) and Cs₂CO₃ (253 mg, 0.78 mmol) in DMF is stirred at room temperature for 16 h and partitioned between water and ethyl acetate. The phases are separated. The organic phase is dried over MgSO₄ and concentrated in vacuo. The resultant residue is purified by flash chromatography with EtOAc/hexane (50:50) to afford the title product as a white solid, 52 mg (48% yield); NMR (400 MHz, DMSO-d₆): δ 2.0 (m, 2H), 2.6 (t, 2H), 4.0 (t, 2H), 5.0 (s, 2H), 5.50 (d, 1H), 5.78 (s, 2H), 6.15 (d, 2H), 6.48 (s, 1H), 6.70 (d, 1H), 6.93 (s, 1H), 7.10 (t, 1H), 7.30-7.50 (m, 4H), 7.53 (d, 1H); MS (+) ES: 417 (M+H)⁺.

### EXAMPLES 9-15

### Preparation of 6-{[6-(Substituted-alkoxy)-2-(2-chlorophenyl)-1H-indol-1-yl]methyl}pyridin-2-amine Compounds

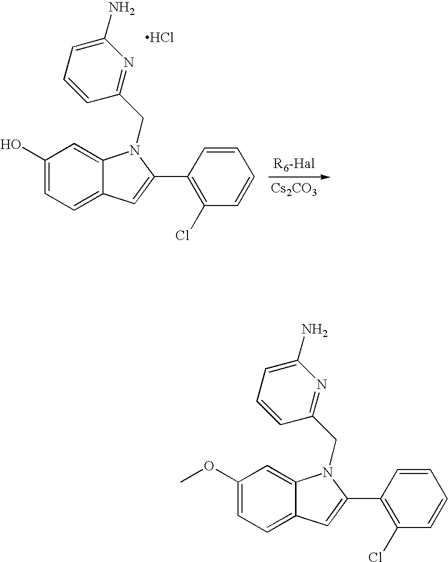

Using essentially the same procedure described in Example 8 and employing the desired alkylhalide, the compounds shown in Table I are obtained and identified by NMR and mass spectral analyses.

TABLE I

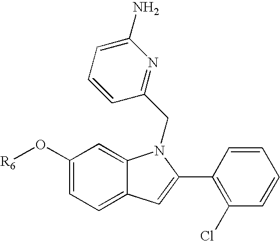

| Ex. | | mp |
|---|---|---|
| No. | R6 | ° C. |
| 9 | benzyl | 64-66 |
| 10 | 3-cyanobenzyl | 65-67 |
| 11 | 3-fluorobenzyl | 60-64 |
| 12 | 2,5-difluorobenzyl | 65-68 |
| 13 | 3-(trifluoromethyl)benzyl | 60-62 |
| 14 | 3-methoxybenzyl | 67-69 |
| 15 | propyl | 128-130 |
| 16 | methyl | 174-176 |

### EXAMPLE 17

### Preparation of 6-{[2-(2-chlorophenyl)-6-(pyrimidin-5-yloxy)-1H-indol-1-yl]methyl}pyridin-2-amine

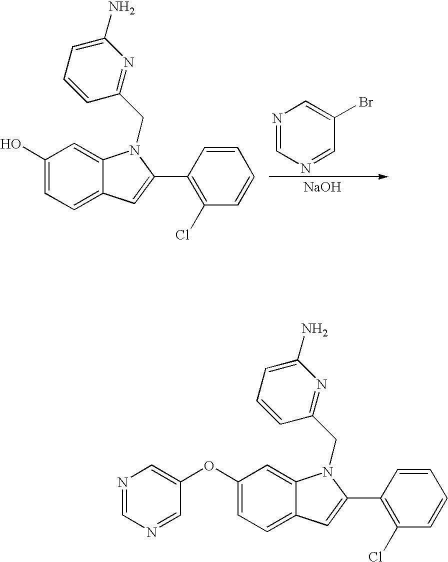

A solution of NaOH (8.0 mg, 0.2 mmol) and 1-[(6-aminopyridin-2-yl)methyl]-2-(2-chlorophenyl)-1H-indol-6-ol (70 mg, 0.20 mmol) in ethanol is stirred for 1 h and concentrated in vacuo. The resultant residue is treated with 5-bromo-pyrimidine (31.6 mg, 0.20 Mmol) and HMPA (1 mL), heated at 150° C. in a microwave oven for 4 min., cooled and chromatographed using column chromatography (silica gel and ethyl acetate as eluent) to afford the title product as a white solid, 20 mg (24% yield), mp 115-117° C., identified by NMR and mass spectral analyses. MS (+) ES: 428 (M+H)⁺.

### EXAMPLE 18

### Preparation of 6-Bromo-2-(2-chlorophenyl)-1H-indole [A] and 4-Bromo-2-(2-chlorophenyl)-1H-indole [B]

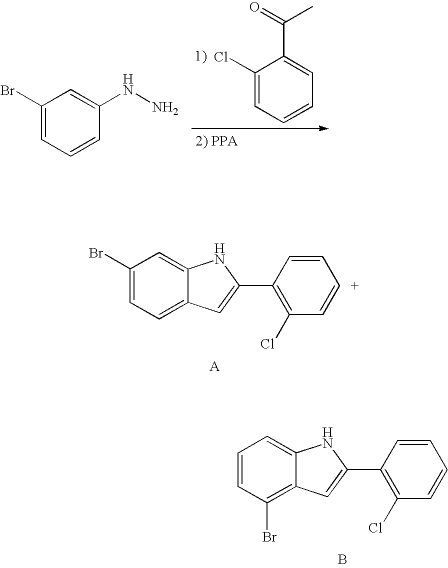

A solution of 3-methoxyphenylhydrazine (1.84 g, 10 mmol) and 2-choloroacetophone (1.54 g, 10 mmol) in ethanol is heated at reflux temperature for 24 h, cooled to room temperature and concentrated under reduced pressure. The resultant residue is flash chromatographed on silica gel with ethyl acetate to afford (1E)-1-(2-chlorophenyl)ethanone(3-bromophenyl)hydrazone as an oil, 3.0 g (93% yield). The oil (3.0 g, 9.2 mMol) is mixed neat with 24 g of PPA, heated at 140° C. for 30 min with stirring, cooled to room temperature, diluted with water and extracted with methelene chloride. The extracts are combined, washed with water, dried over MgSO₄ and concentrated in vacuo. The resultant residue is purified by flash chromatography with ethyl acetate/hexane to afford a mixture of the title products as an off-white solid. The mixture was separated using flash chromatography to give: Title product A as a white solid, identified by NMR and mass spectral analyses; and Title product B as a white solid, identified by NMR and mass spectral analyses.

### EXAMPLE 19

### Preparation of 6-{[6-Bromo-2-(2-chlorophenyl)-1H-indol-1-yl]methyl}pyridin-2-amine

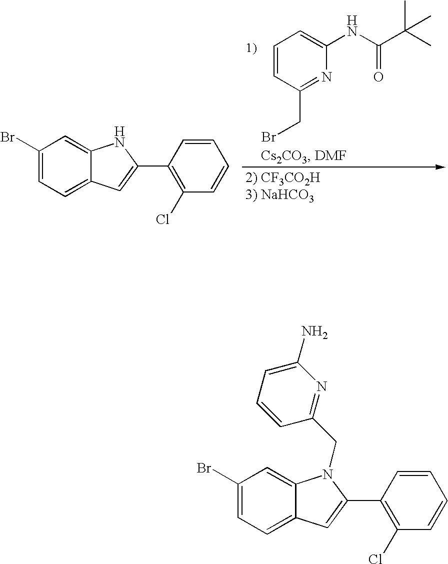

Using essentially the same procedure described in Example 5 and employing 6-bromo-2-(2-chlorophenyl)-1H-indole and N-(6-bromomethylpyridin-2-yl)-2,2-dimethyl-propionamide, the N-(6-{[6-bromo-2-(2-chlorophenyl)-1H-indol-1-yl]methyl}pyridine-2-yl)-2,2-dimethylpropanamide intermediate is obtained. Said intermediate (170 mg, 0.33 mmol) is treated with trifluoroacetic acid (5 mL) and methylene chloride, stirred at room temperature for 4 h and concentrated to dryness to afford the trifluoroacetic acid salt of the title product as a tan solid, 138 mg (82% yield). The tan solid is dissolved in methylene chloride, washed with saturated sodium bicarbonate and concentrated in vacuo to afford the title product as a white solid, mp 189-191° C., identified by NMR and mass spectral analyses. MS (+) ES: 412 (M+H)⁺.

### EXAMPLE 20

### Preparation of 4-{1-[(6-Aminopyridin-2-yl)methyl]-2-(2-chlorophenyl)-1H-indol-6-yl}but-3-yn-1-ol

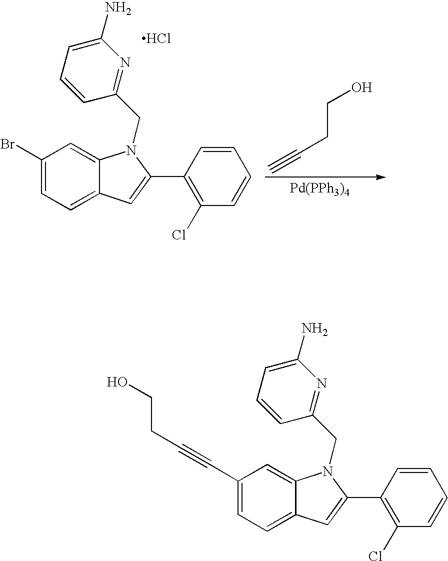

A mixture of 6-{[6-bromo-2-(2-chlorophenyl)-1H-indol-1-yl]methyl}pyridin-2-amine hydrochloride (100 mg, 0.22 mmol), 1-butyne-4-ol (46 mg, 0.66 mmol) and Pd(PPh₃)₄ (25 mg, 0.022 mmol) in pyrrolidine is heated at 75° C. under a nitrogen atmosphere for 5 h, diluted with water and extracted twice with CH₂Cl₂. The extracts are combined and concentrated to dryness to give a residue. The residue is purified by chromatography on a silica cartridge to give 88 mg of a yellow oil. The oil is further purified by preparative HPLC. The purified material is treated with water, basified with aqueous NaHCO₃ and extracted with CH₂Cl₂ to give the title product, 25 mg (28% yield), identified by NMR and mass spectral analyses. NMR (400 MHz, DMSO-d₆): δ 7.64-7.34 (m, 6H); 7.14 (dd, 1H); 7.09 (dd, 1H); 6.59 (d, 1H); 6.22 (d, 1H); 5.84 (s, 2H); 5.62 (d, 1H); 5.03 (s, 2H); 4.83 (t, 1H); 3.57 (dt, 2H); 2.54 (t, 2H).

### EXAMPLE 21

### Preparation of 6-{[6-(4-fluorobutyn-1-yl)-2-(2-chlorophenyl)-1H-indol-1-yl]methyl}pyridin-2-amine

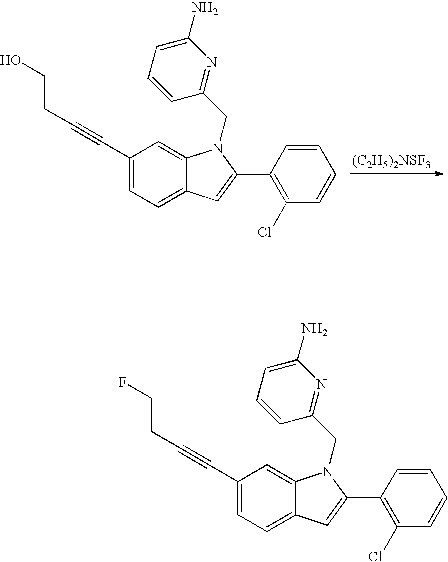

A solution of 4-{1-[(6-aminopyridin-2-yl)methyl]-2-(2-chlorophenyl)-1H-indol-6-yl}but-3-yn-1-ol (393 mg, 0.98 mmol) in dry CH₂Cl₂, under a nitrogen atmosphere, is cooled to −70° C.; treated with diaminosulfurtrifluoride (DAST) (174 mg, 1.08 mmol), slowly warmed to room temperature over a 3 h period, stirred overnight at room temperature and concentrated in vacuo. The residue is purified first by a flash chromatography on a silica cartridge, then by preparative HPLC, to obtain the title product as a white solid, 34 mg (9% yield), identified by NMR and mass spectral analyses.

### EXAMPLE 22

### Preparation of 5-{1-[(6-Aminopyridin-2-yl)methyl]-2-(2-chlorophenyl)-1H-indol-6-yl}pent-4-yn-1-ol

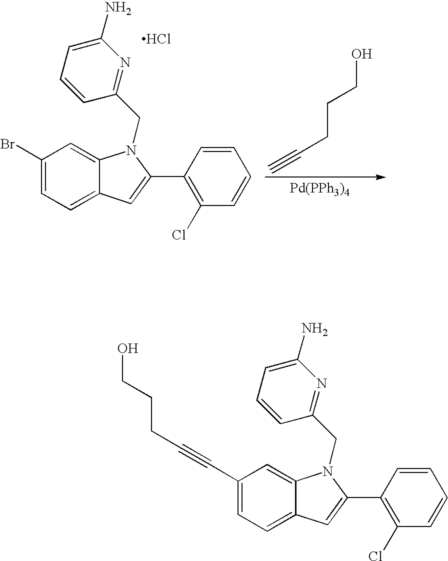

Using essentially the same procedure described in Example 20 and employing 1-pentyn-5-ol, the title compound is obtained and identified by NMR and mass spectral analyses. NMR (400 MHz, DMSO-d₆): δ 7.63-7.34 (m, 6H); 7.14 (dd, 1H); 7.08 (dd, 1H); 6.59 (d, 1H); 6.22 (d, 1H); 5.83 (s, 2H); 5.61 (d, 1H); 5.03 (s, 2H); 4.48 (t, 1H); 3.51 (dt, 2H); 2.44 (t, 2H); 1.68 (m, 2H).

### EXAMPLE 23

### Preparation of 6-{1-[(6-Aminopyridin-2-yl)methyl]-2-(2-chlorophenyl)-1H-indol-6-yl}hex-5-yn-1-ol

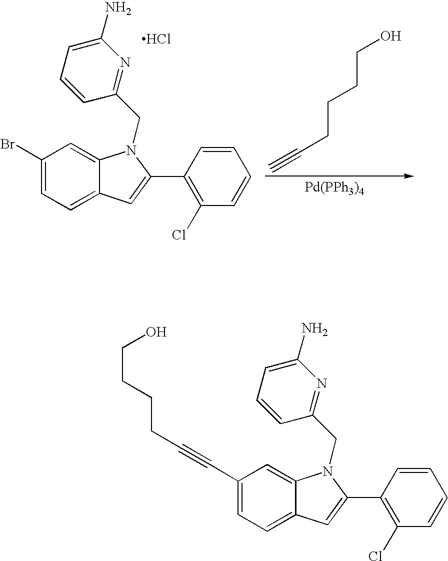

Using essentially the same procedure described in Example 20 and employing 1-hexyn-6-ol, the title compound is obtained and identified by NMR and mass spectral analyses. NMR (400 MHz, DMSO-d₆): δ 7.61 (dd, 1H); 7.57 (dd, 1H); 7.54-7.36 (m, 4H); 7.15 (dd, 1H); 7.08 (dd, 1H); 6.59 (d, 1H); 6.22 (dd, 1H); 5.83 (s, 2H); 5.62 (d, 1H); 5.04 (s, 2H); 4.38 (t, 1H); 3.44 (m, 2H); 2.41 (m, 2H); 1.57 (m, 4H).

### EXAMPLE 24

### Preparation of 4-[1-(6-Aminopyridin-2-ylmethyl)-2-(2-chlorophenyl)-1H-indol-6-yl]butan-1-ol

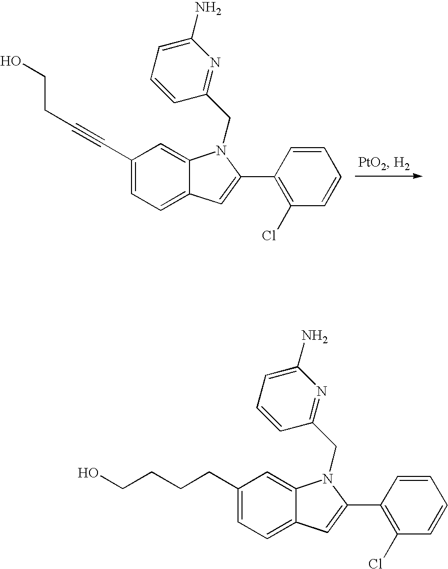

A mixture of 4-{1-[(6-aminopyridin-2-yl)methyl]-2-(2-chlorophenyl)-1H-indol-6-yl}but-3-yn-1-ol (98 mg, 0.244 mmoles) and PtO₂ (15 mg) in ethanol is hydrogenated at 15 psi in a Parr hydrogenator for 4 hours. The reaction mixture is filtered through a celite pad. The filtrate is concentrated under reduced pressure. The resultant residue (quantitative yield) is purified using preparative HPLC. The purified material is taken up in water, basified with saturated NaHCO₃ and extracted with CH₂Cl₂. The extracts are combined and evaporated to dryness to give the title compound, 40.3 mg (40% yield), identified by NMR and mass spectral analyses. NMR (400 MHz, DMSO-d₆): δ 7.59 (dd, 1H); 7.54-7,32 (m, 4H); 7.17 (s, 1H); 7.12 (dd, 1H); 6.93 (dd, 1H); 6.51 (d, 1H); 6.20 (d, 1H); 5.81 (s, 2H); 5.57 (d, 1H); 5.00 (s, 2H); 4.30 (t, 1H); 3.39 (m,

### EXAMPLE 25

### Preparation of 5-[1-(6-Aminopyridin-2-ylmethyl)-2-(2-chlorophenyl)-1H-indol-6-yl]pentan-1-ol

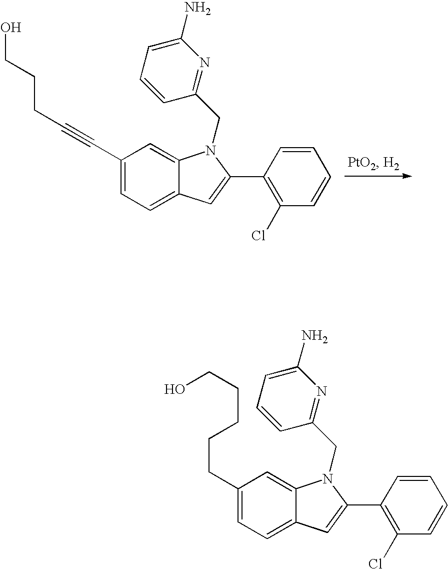

Using essentially the same procedure described in Example 24 and employing 5-{1-[(6-aminopyridin-2-yl)methyl]-2-(2-chlorophenyl)-1H-indol-6-yl}pent-4-yn-1-ol as substrate, the title compound is obtained and identified by NMR and mass spectral analyses. NMR (400 MHz, DMSO-d₆): δ 7.59 (dd, 1H); 7.54-7,24 (m, 4H); 7.16 (s, 1H); 7.12 (dd, 1H); 6.93 (dd, 1H); 6.51 (d, 1H); 6.20 (d, 1H); 5.82 (s, 2H); 5.58 (d, 1H); 5.00 (s, 2H); 4.28 (m, 1H); 3,36 (m, 2H); 2,63 (m, 2H); 1.64-1.17 (m, 6H).

### EXAMPLE 26

### Preparation of 6-[1-(6-Aminopyridin-2-ylmethyl)-2-(2-chlorophenyl)-1H-indol-6-yl]hexan-1-ol

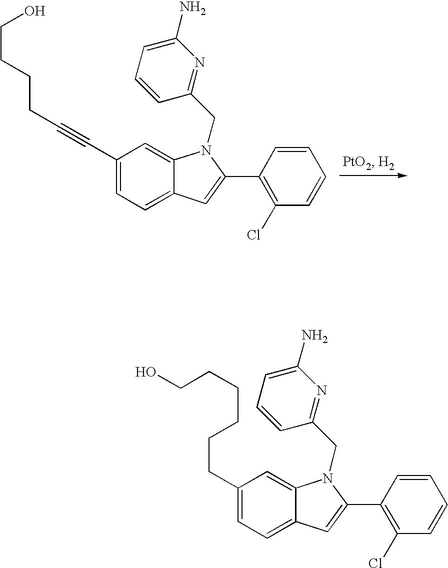

Using essentially the same procedure described in Example 24 and employing 6-{1-[(6-aminopyridin-2-yl)methyl]-2-(2-chlorophenyl)-1H-indol-6-yl}hex-5-yn-1-ol as substrate, the title compound is obtained and identified by NMR and mass spectral analyses. NMR (400 MHz, DMSO-d₆): δ 7.58 (dd, 1H); 7.53-7,34 (m, 4H); 7.16 (s, 1H); 7.12 (dd, 1H); 6.93 (dd, 1H); 6.51 (d, 1H); 6.20 (d, 1H); 5.80 (s, 2H); 5.58 (d, 1H); 5.00 (s, 2H); 4.27 (t, 1H); 3.36 (m, 2H); 2.62 (t, 2H); 1.57 (m, 2H); 1.44-1.18 (m, 6H).

### EXAMPLE 27

### Preparation of 6-{[2-(2-Chlorophenyl)-6-(4-methoxybut-1-yn-1-yl)-1H-indol-1-yl]methyl}pyridin-2-amine

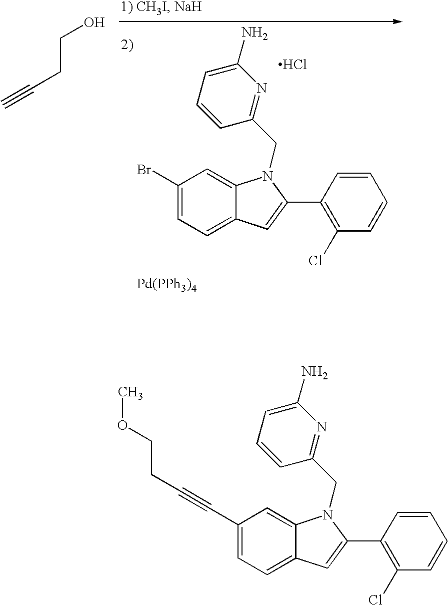

A cooled mixture of 60% NaH (270 mg, 6.7 mmol) in DMF; under a nitrogen atmosphere, is treated with butynol (468 mg, 6.67 mmol), stirred for after 30 min, treated with CH₃I (946 mg, 6.67 mmol), warmed at 40° C. for 1.5 h in a closed vessel. The reaction mixture is treated with 6-{[6-bromo-2-(2-chlorophenyl)-1H-indol-1-yl]methyl}pyridin-2-amine hydrochloride (400 mg, 0.89 mmol), pyrrolidine (6 ml), and Pd-tetrakis (102 mg, 0.089 mmol), heated at 70° C. under nitrogen for 3 h, treated with another portion of Pd-tetrakis (102 mg, 0.089 mmol) and heated at 70° C. for 16 h (LC-MS shows an approximately 1:1 mixture of desired product and OH-derivative). The reaction mixture is concentrated under reduced pressure to remove the pyrrolidine. The resultant residue is diluted with water and extracted with CH₂Cl₂. The extracts are combined, washed sequentially with water and brine, dried over MgSO₄ and evaporated to dryness. This residue is purified by a flash chromatography on a silica cartridge, then by preparative HPLC, to obtain the title product as a yellow solid, 52 mg (yield: 14%), identified by NMR and mass spectral analyses. NMR (400 MHz, DMSO-d₆): δ 7.67-7.34 (m, 6H); 7.18 (dd, 1H); 7.09 (dd, 1H); 6.60 (d, 1H); 6.26 (d, 1H); 5.97 (s br, 2H); 5.63 (d, 1H); 5.06 (s, 2H); 3.51 (t, 2H); 3.29 (s, 3H); 2.65 (t, 2H).

### EXAMPLE 28

### Preparation of 6-{[2-(2-Chlorophenyl)-6-(5-methoxypent-1-yn-1-yl)-1H-indol-1-yl]methyl}pyridin-2-amine

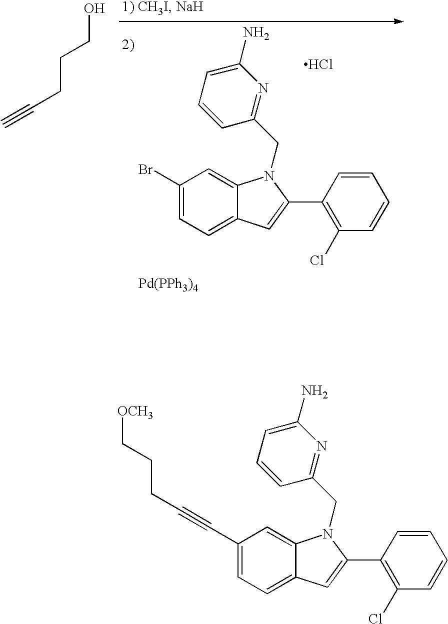

Using essentially the same procedure described in Example 27 and employing 1-pentyn-5-ol, the title compound is obtained and identified by NMR and mass spectral analyses.

### EXAMPLE 29

### Preparation of 6-{[2-(2-Chlorophenyl)-6-(5-methoxyhex-1-yn-1-yl)-1H-indol-1-yl]methyl}pyridin-2-amine

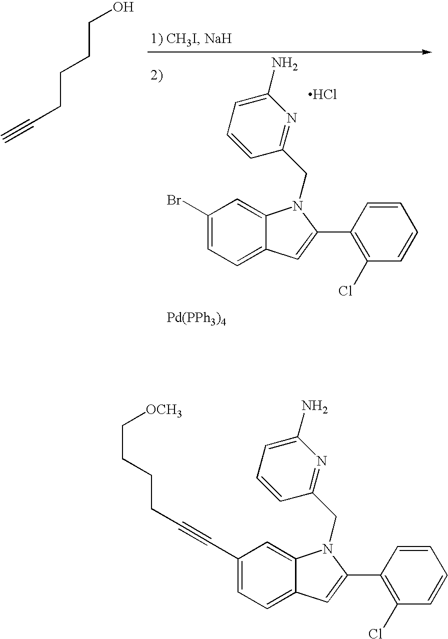

Using essentially the same procedure described in Example 27 and employing 1-hexyn-5-ol, the title compound is obtained and identified by NMR and mass spectral analyses.

### EXAMPLE 30

### Preparation of 6-[(2-Phenyl)-1H-indol-1-yl)methyl]pyridin-2-amine

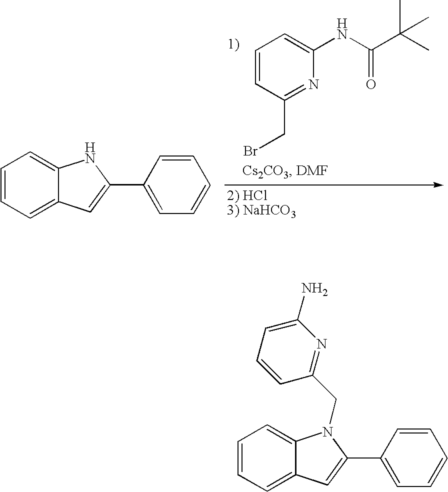

Using essentially the same procedures described in Examples 5 and 7 and employing 2-phenyl-1H-indole as substrate, the title compound is obtained as a white solid, mp 45-47° C., identified by NMR and mass spectral analyses. MS (+) ES: 300 (M+H)⁺.

### EXAMPLE 31

### Preparation of 6-{[2-(2-chlorophenyl)-6-(5-fluoropent-1-yn-1-yl)-1H-indol-1-yl]methyl}pyridin-2-amine

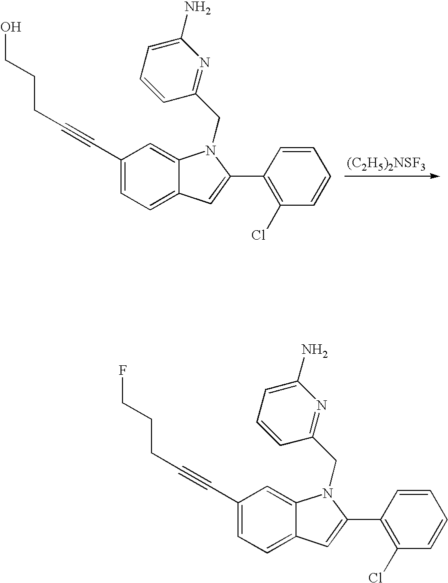

Using essentially the same procedure described in Example 21 and employing 5-{1-[(6-aminopyridin-2-yl)methyl]-2-(2-chlorophenyl)-1H-indol-6-yl}pent-4-yn-1-ol, the title compound is obtained and identified by NMR and mass spectral analyses.

### EXAMPLE 32

### Preparation of 6-{[2-(2-chlorophenyl)-6-(6-fluorohex-1-yn-1-yl)-1H-indol-1-yl]methyl}pyridin-2-amine

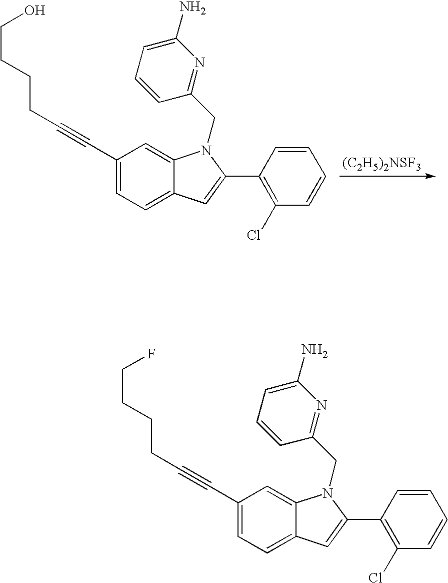

Using essentially the same procedure described in Example 21 and employing 6-{1-[(6-aminopyridin-2-yl)methyl]-2-(2-chlorophenyl)-1H-indol-6-yl}hex-5-yn-1-ol, the title compound is obtained and identified by NMR and mass spectral analyses.

### EXAMPLE 33

### Evaluation of BACE1 Binding Affinity of Test Compounds

Fluorescent Kinetic Assays

Final Assay Conditions: 10 nM human BACE1 (or 10 nM Murine BACE1, 1.5 nM human BACE2), 25 μM substrate (WABC-6, MW 1549.6, from AnaSpec), Buffer: 50 mM Na-Acetate, pH 4.5, 0.05% CHAPS, 25% PBS, room temperature. Na-Acetate was from Aldrich, Cat.# 24, 124-5, CHAPS was from Research Organics, Cat. # 1304C 1×, PBS was from Mediatech (Cellgro), Cat# 21-031-CV, peptide substrate AbzSEVNLDAEFRDpa was from AnaSpec, Peptide Name: WABC-6

Determination of stock substrate (AbzSEVNLDAEFRDpa) concentration: ˜25 mM stock solution is made in DMSO using the peptide weight and MW, and diluted to ˜25 μM (1:1000) in 1×PBS. Concentration is determined by absorbance at 354 nm using an extinction coefficient ε of 18172 M⁻¹cm⁻¹, the concentration of stock substrate is corrected, and the substrate stock stored in small aliquots in −80° C.
[Substrate Stock]=ABS^{354 nm}*10⁶/18172 (in mM)
The extinction coefficient ε^{354 nm} was adapted from TACE peptide substrate, which had the same quencher-fluorophore pair.
Determination of Stock Enzyme Concentration: the stock concentration of each enzyme is determined by absorbance at 280 nm using an ε of 64150 M⁻¹cm⁻¹ for hBACE1 and MuBACE1, 62870 M⁻¹cm⁻¹ for hBACE2 in 6 M Guanidinium Hydrochloride (from Research Organics, Cat. # 5134G-2), pH ˜6. The extinction coefficient ε^{280 nm} for each enzyme was calculated based on known amino acid composition and published extinction coefficients for Trp (5.69 M⁻¹ cm⁻¹) and Tyr (1.28 M⁻¹ cm⁻¹) residues (Anal. Biochem. 182, 319-326).
Dilution and mixing steps: total reaction volume: 100 μL

2× inhibitor dilutions in buffer A (66.7 mM Na-Acetate, pH 4.5, 0.0667% CHAPS) were prepared,

4× enzyme dilution in buffer A (66.7 mM Na-Acetate, pH 4.5, 0.0667% CHAPS) were prepared,

100 μM substrate dilution in 1×PBS was prepared, and

50 μL 2× Inhibitor, 25 μL 100 μM substrate are added to each well of 96-well plate (from DYNEX Technologies, VWR #: 11311-046), immediately followed by 25 μL 4× enzyme (added to the inhibitor and substrate mix), and the fluorescence readings are initiated.

Fluorescence Readings: Readings at λₑₓ 320 nm and λₑₘ 420 nm are taken every 40 sec for 30 min at room temperature and the linear slope for substrate cleavage rate (vᵢ) determined.

Calculation of % Inhibition:
% Inhibition=100*(1−vᵢ/v₀)
vᵢ: substrate cleavage rate in the presence of inhibitor
v₀: substrate cleavage rate in the absence of inhibitor
IC₅₀ Determination:
% Inhibition=((B*IC₅₀ⁿ)+(100*I₀ⁿ))/(IC₅₀ⁿ+I₀ⁿ)
(Model # 39 from LSW Tool Bar in Excel where B is the % inhibition from the enzyme control, which should be close to 0.) % Inhibition is plotted vs. Inhibitor Concentration (I₀) and the data fit to the above equation to obtain IC₅₀ value and Hill number (n) for each compound. Testing at least 10 different inhibitor concentrations is preferred.

The data obtained are shown in Table II.

TABLE II

| Example | BACE-1 |
|---|---|
| No. | % Inhibition at 5 μM |
| 7 | — |
| 8 | 79 |
| 9 | 31.6 |
| 10 | 58.0 |
| 11 | 49.5 |
| 12 | 33.2 |
| 13 | 13.8 |
| 14 | 36.3 |
| 15 | 38.6 |
| 16 | 30.1 |
| 17 | 39.8 |
| 19 | 31.7 |
| 20 | 67.5 |
| 21 | 56.6 |
| 22 | 71.2 |
| 23 | 81.1 |
| 24 | 71.8 |
| 25 | 77.3 |
| 26 | 106.7 |
| 27 | 51.0 |
| 28 | 53.0 |
| 29 | 53.9 |
| 30 | 24.6 |
| 31 | 64.6 |
| 32 | 53.4 |

What is claimed is:

1. A compound of formula I 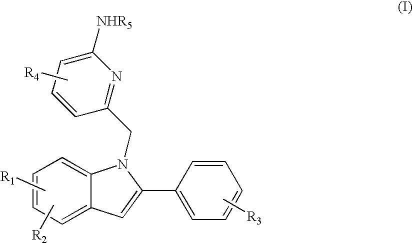
wherein
R₁ and R₂ are each independently H, halogen, CN, OR₆, CO₂R₇, COR₈, NR₁₁R₁₂ or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₃ is H, halogen or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₄ is H, halogen, NR₁₃R₁₄, OR₁₅ or a C₁-C₆alkyl or aryl group each group optionally substituted;
R₅ is H or an optionally substituted C₁-C₆alkyl group;
R₆ is H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₇, R₈ and R₁₅ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted; and
R₁₁, R₁₂, R₁₃ and R₁₄ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted or R₁₁R₁₂ or R₁₃R₁₄ may be taken together with the atom to which they are attached to form an optionally substituted 5- to 7-membered ring optionally containing an additional heteroatom selected from O, N or S; or
a stereoisomer thereof or a pharmaceutically acceptable salt thereof.

2. The compound according to claim 1 wherein R₄ and R₅ are H.

3. The compound according to claim 1 wherein R₃ is H or halogen.

4. The compound according to claim 1 wherein R₁ is OR₆ and R₂ is H.

5. The compound according to claim 2 wherein R₃ is halogen and R₃ is in the 2-position.

6. The compound according to claim 2 wherein R₁ is OR₆ and R₆ is a C₁-C₆alkyl, C₂-C₆alkenyl, C₂-C₆alkynyl or heteroaryl group each group optionally substituted.

7. The compound according to claim 5 wherein R₁ is OR₆ and R₂ is H.

8. The compound according to claim 7 wherein R₆ is a C₁-C₆alkyl, C₂-C₆alkenyl, C₂-C₆alkynyl or heteroaryl group each group optionally substituted.

9. The compound according to claim 1 selected from the group consisting essentially of:
4-{[1-[(6-aminopyridin-2-yl)methyl]-2-(2-chlorophenyl)-1H-indol-6-yl]oxy}butanenitrile;
6-{[6-bromo-2-(2-chlorophenyl)-1H-indol-1-yl]methyl}pyridin-2-amine;
6-{[6-(benzyloxy)-2-(2-chlorophenyl)-1H-indol-1-yl]methyl}pyridin-2-amine;
6-({2-(2-chlorophenyl)-6-[(3-fluorobenzyl)oxy]-1H-indol-1-yl}methyl)pyridin-2-amine;
3-({[1-[(6-aminopyridin-2-yl)methyl]-2-(2-chlorophenyl)-1H-indol-6-yl]oxy}methyl)benzonitrile;
6-({2-(2-chlorophenyl)-6-[(2,5-difluorobenzyl)oxy]-1H-indol-1-yl}methyl)pyridin-2-amine;
6-[(2-(2-chlorophenyl)-6-{[3-(trifluoromethyl)benzyl]oxy}-1H-indol-1-yl)methyl]pyridin-2-amine;
6-({2-(2-chlorophenyl)-6-[(3-methoxybenzyl)oxy]-1H-indol-1-yl}methyl)pyridin-2-amine;
6-{[2-(2-chlorophenyl)-6-(pyrimidin-5-yloxy)-1H-indol-1-yl]methyl}pyridin-2-amine;
6-{[2-(2-chlorophenyl)-6-propoxy-1H-indol-1-yl]methyl}pyridin-2-amine;
6-{[2-(2-chlorophenyl)-6-methoxy-1H-indol-1-yl]methyl}pyridin-2-amine;
6-[(2-phenyl-1H-indol-1-yl)methyl]pyridin-2-amine;
4-[1-[(6-aminopyridin-2-yl)methyl]-2-(2-chlorophenyl)-1H-indol-6-yl]but-3-yn-1-ol;
5-[1-[(6-aminopyridin-2-yl)methyl]-2-(2-chlorophenyl)-1H-indol-6-yl]pent-4-yn-1-ol;
6-[1-[(6-aminopyridin-2-yl)methyl]-2-(2-chlorophenyl)-1H-indol-6-yl]hex-5-yn-1-ol;
4-[1-[(6-aminopyridin-2-yl)methyl]-2-(2-chlorophenyl)-1H-indol-6-yl]butan-1-ol;
5-[1-[(6-aminopyridin-2-yl)methyl]-2-(2-chlorophenyl)-1H-indol-6-yl]pentan-1-ol;
6-[1-[(6-aminopyridin-2-yl)methyl]-2-(2-chlorophenyl)-1H-indol-6-yl]hexan-1-ol;
6-{[2-(2-chlorophenyl)-6-(4-methoxybut-1-yn-1-yl)-1H-indol-1-yl]methyl}pyridine-2-amine;
1-[(6-aminopyridin-2-yl)methyl]-2-(2-chlorophenyl)-1H-indol-6-ol;
6-{[2-(2-chlorophenyl)-6-(6-methoxyhex-1-yn-1-yl)-1H-indol-1-yl]methyl}pyridin-2-amine;
6-{[2-(2-chlorophenyl)-6-(5-fluoropent-1-yn-1-yl)-1H-indol-1-yl]methyl}pyridin-2-amine;
6-{[2-(2-chlorophenyl)-6-(6-fluorohex-1-yn-1-yl)-1H-indol-1-yl]methyl}pyridin-2-amine;
6-{[2-(2-chlorophenyl)-6-(5-methoxypent-1-yn-1-yl)-1H-indol-1-yl]methyl}pyridin-2-amine;
6-{[2-(2-chlorophenyl)-6-(4-fluorobut-1-yn-1-yl)-1H-indol-1-yl]methyl}pyridin-2-amine;
a stereoisomer thereof; and
a pharmaceutically acceptable salt thereof.

10. A method for the treatment of a disease or disorder associated with excessive BACE activity in a patient in need thereof which comprises providing to said patient a therapeutically effective amount of a compound of formula I 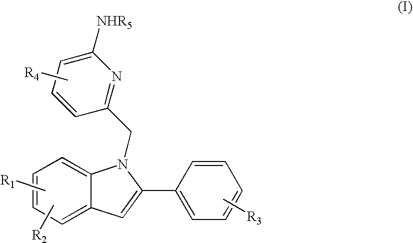
wherein
R₁ and R₂ are each independently H, halogen, CN, OR₆, CO₂R₇, COR₈, NR₁₁R₁₂ or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₃ is H, halogen or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₄ is H, halogen, NR₁₃R₁₄, OR₁₅ or a C₁-C₆alkyl or aryl group each group optionally substituted;
R₅ is H or an optionally substituted C₁-C₆alkyl group;
R₆ is a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₇, R₈ and R₁₅ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted; and
R₁₁, R₁₂, R₁₃ and R₁₄ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted or R₁₁R₁₂ or R₁₃R₁₄ may be taken together with the atom to which they are attached to form an optionally substituted 5- to 7-membered ring optionally containing an additional heteroatom selected from O, N or S; or
a stereoisomer thereof or a pharmaceutically acceptable salt thereof.

11. The method according to claim 10 wherein said disease or disorder is selected from the group consisting of: Alzheimer's disease; cognitive impairment; Down's Syndrome; HCHWA-D; cognitive decline; senile dementia; cerebral amyloid angiopathy; and a neurodegenerative disorder.

12. The method according to claim 10 wherein said disease or disorder is characterized by the production of β-amyloid deposits or neurofibrillary tangles.

13. A method for modulating the activity of BACE which comprises contacting a receptor thereof with an effective amount of a compound according to claim 1 wherein R₆ is other than H.

14. A method for the treatment of Alzheimer's disease in a patient in need thereof which comprises providing to said patient an effective amount of a compound according to claim 1 wherein R₆ is other than H.

15. A pharmaceutical composition which comprises a pharmaceutically acceptable carrier and an effective amount of a compound of formula I 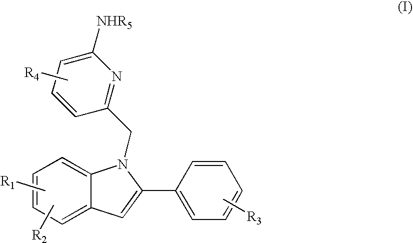
wherein
R₁ and R₂ are each independently H, halogen, CN, OR₆, CO₂R₇, COR₈, NR₁₁R₁₂ or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₃ is H, halogen or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₄ is H, halogen, NR₁₃R₁₄, OR₁₅ or a C₁-C₆alkyl or aryl group each group optionally substituted;
R₅ is H or an optionally substituted C₁-C₆alkyl group;
R₆ is a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₇, R₈ and R₁₅ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted; and
R₁₁, R₁₂, R₁₃ and R₁₄ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted or R₁₁R₁₂ or R₁₃R₁₄ may be taken together with the atom to which they are attached to form an optionally substituted 5- to 7-membered ring optionally containing an additional heteroatom selected from O, N or S; or
a stereoisomer thereof or a pharmaceutically acceptable salt thereof.

16. The composition according to claim 15 having a formula I compound wherein R₄ and R₅ are H.

17. The composition according to claim 16 having a formula I compound wherein R₃ is halogen and is in the 2-position.

18. The composition according to claim 17 having a formula I compound wherein R₁ is OR₆; R₂ is H; and R₆ is a C₁-C₆alkyl, C₂-C₆alkenyl, C₂-C₆alkynyl or heteroaryl group each group optionally substituted.

19. The composition according to claim 15 having a formula I compound selected from the group consisting essentially of:
4-{[1-[(6-aminopyridin-2-yl)methyl]-2-(2-chlorophenyl)-1H-indol-6-yl]oxy}butanenitrile;
6-{[6-bromo-2-(2-chlorophenyl)-1H-indol-1-yl]methyl}pyridin-2-amine;
6-{[6-(benzyloxy)-2-(2-chlorophenyl)-1H-indol-1-yl]methyl}pyridin-2-amine;
6-({2-(2-chlorophenyl)-6-[(3-fluorobenzyl)oxy]-1H-indol-1-yl}methyl)pyridin-2-amine;
3-({[1-[(6-aminopyridin-2-yl)methyl]-2-(2-chlorophenyl)-1H-indol-6-yl]oxy}methyl)benzonitrile;
6-({2-(2-chlorophenyl)-6-[(2,5-difluorobenzyl)oxy]-1H-indol-1-yl}methyl)pyridin-2-amine;
6-[(2-(2-chlorophenyl)-6-{[3-(trifluoromethyl)benzyl]oxy}-1H-indol-1-yl)methyl]pyridin-2-amine;
6-({2-(2-chlorophenyl)-6-[(3-methoxybenzyl)oxy]-1H-indol-1-yl}methyl)pyridin-2-amine;
6-{[2-(2-chlorophenyl)-6-(pyrimidin-5-yloxy)-1H-indol-1-yl]methyl}pyridin-2-amine;
6-{[2-(2-chlorophenyl)-6-propoxy-1H-indol-1-yl]methyl}pyridin-2-amine;
6-{[2-(2-chlorophenyl)-6-methoxy-1H-indol-1-yl]methyl}pyridin-2-amine;
6-[(2-phenyl-1H-indol-1-yl)methyl]pyridin-2-amine;
4-[1-[(6-aminopyridin-2-yl)methyl]-2-(2-chlorophenyl)-1H-indol-6-yl]but-3-yn-1-ol;
5-[1-[(6-aminopyridin-2-yl)methyl]-2-(2-chlorophenyl)-1H-indol-6-yl]pent-4-yn-1-ol;
6-[1-[(6-aminopyridin-2-yl)methyl]-2-(2-chlorophenyl)-1H-indol-6-yl]hex-5-yn-1-ol;
4-[1-[(6-aminopyridin-2-yl)methyl]-2-(2-chlorophenyl)-1H-indol-6-yl]butan-1-ol;
5-[1-[(6-aminopyridin-2-yl)methyl]-2-(2-chlorophenyl)-1H-indol-6-yl]pentan-1-ol;
6-[1-[(6-aminopyridin-2-yl)methyl]-2-(2-chlorophenyl)-1H-indol-6-yl]hexan-1-ol;
6-{[2-(2-chlorophenyl)-6-(4-methoxybut-1-yn-1-yl)-1H-indol-1-yl]methyl}pyridine-2-amine;
1-[(6-aminopyridin-2-yl)methyl]-2-(2-chlorophenyl)-1H-indol-6-ol;
6-{[2-(2-chlorophenyl)-6-(6-methoxyhex-1-yn-1-yl)-1H-indol-1-yl]methyl}pyridin-2-amine;
6-{[2-(2-chlorophenyl)-6-(5-fluoropent-1-yn-1-yl)-1H-indol-1-yl]methyl}pyridin-2-amine;
6-{[2-(2-chlorophenyl)-6-(6-fluorohex-1-yn-1-yl)-1H-indol-1-yl]methyl}pyridin-2-amine;
6-{[2-(2-chlorophenyl)-6-(5-methoxypent-1-yn-1-yl)-1H-indol-1-yl]methyl}pyridin-2-amine;
6-{[2-(2-chlorophenyl)-6-(4-fluorobut-1-yn-1-yl)-1H-indol-1-yl]methyl}pyridin-2-amine;
a stereoisomer thereof; and
a pharmaceutically acceptable salt thereof.

20. A method for the preparation of a compound of formula Ia 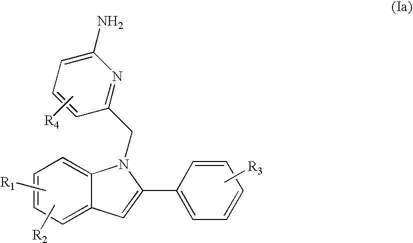
wherein
R₁ and R₂ are each independently H, halogen, CN, OR₆, CO₂R₇, COR₈, NR₁₁R₁₂ or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₃ is H, halogen or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₄ is H, halogen, NR₁₃R₁₄, OR₁₅ or a C₁-C₆alkyl or aryl group each group optionally substituted;
R₅ is H or an optionally substituted C₁-C₆alkyl group;
R₆ is H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted;
R₇, R₈ and R₁₅ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted; and
R₁₁, R₁₂, R₁₃ and R₁₄ are each independently H or a C₁-C₆alkyl, C₂-C₆alkenyl, C₂-C₆alkynyl, C₃-C₈cycloalkyl, C₃-C₈cycloheteroalkyl, aryl or heteroaryl group each group optionally substituted or R₁₁R₁₂ or R₁₃R₁₄ may be taken together with the atom to which they are attached to form an optionally substituted 5- to 7-membered ring optionally containing an additional heteroatom selected from O, N or S
which process comprises: reacting a compound of formula II 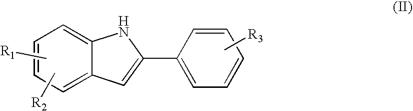
wherein R₁, R₂ and R₃ are as described hereinabove with a compound of formula III 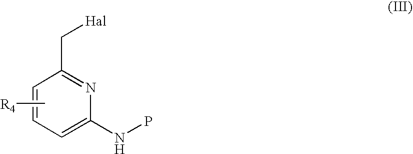
wherein R₄ is as described hereinabove; Hal is Cl, Br or I; and P is a protecting group in the presence of a base optionally in the presence of a solvent to give the protected indolylalkylpyridin-2-amine intermediate; and reacting said intermediate with an acid to give the desired compound of formula Ia.
